(19) United States
(12) Patent Application Publication
(10) Pub. No.: US 2003/0109583 A1
(43) Pub. Date: Jun. 12, 2003

(54) Administration of negamycin or deoxynegamycin for the treatment of bacterial infections

(76) Inventors: Bore G. Raju, Fremont, CA (US); Dinesh V. Patel, Fremont, CA (US); Joaquim Trias, Millbrae, CA (US)

Correspondence Address: Gerald F. Swiss, BURNS, DOANE, SWECKER & MATHIS, L.L.P., P.O. Box 1404, Alexandria, VA, 22313-1404, US

(21) Appl. No.: 10/202,632

(22) Filed: Jul. 25, 2002

Related U.S. Application Data
(60) Provisional application No. 60/308,001, filed on Jul. 25, 2001.

(51) Int. Cl. A61K 31/198

(52) U.S. Cl. 514/563

(57) ABSTRACT

The invention provides a method for treating bacterial infections. In one aspect, the invention comprises orally administering a pharmaceutical composition to an animal, wherein the composition comprises a pharmaceutically acceptable excipient and an antibacterial effective amount of negamycin, or a pharmaceutically acceptable salt, prodrug or isomer thereof. An aspect of the invention also relates to a method of treating a bacterial infection, wherein the method comprises intravenously administering a pharmaceutical composition to an animal, and wherein the composition comprises a pharmaceutically acceptable excipient and an antibacterial effective amount of deoxynegamycin, or a pharmaceutically acceptable salt, prodrug or isomer thereof. An aspect of the invention also relates to a method of treating a bacterial infection, wherein the method comprises administering to an animal an antibacterial effective amount of negamycin or deoxynegamycin, or a pharmaceutically acceptable salt, prodrug or isomer thereof, and wherein the infecting bacteria are selected from a group of bacteria consisting of the following: Acinetobacter baumanii, Citrobacter freundii, Enterobacter aerogenes, haemophilus influenzae, Moraxella catarrhalis, Staphylococcus aureus MRSA, Staphylococcus aureus GISA, Staphylococcus epidermis, Streptococcus pneumoniae PenR, Streptococcus pneumoniae PenS and Streptococcus pyogenes.

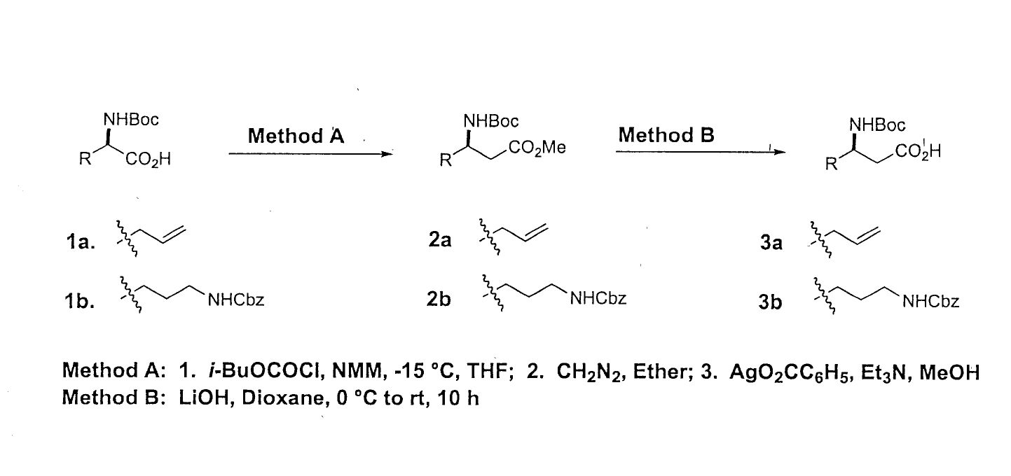

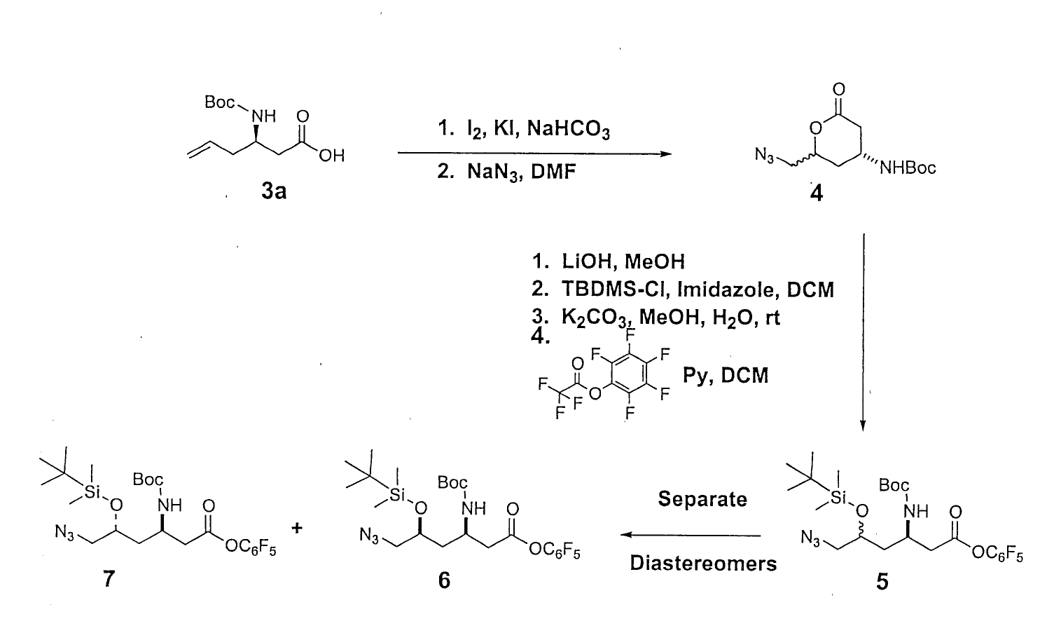

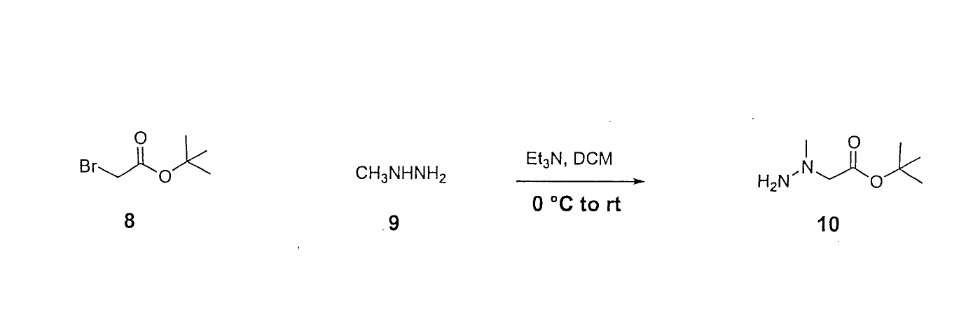

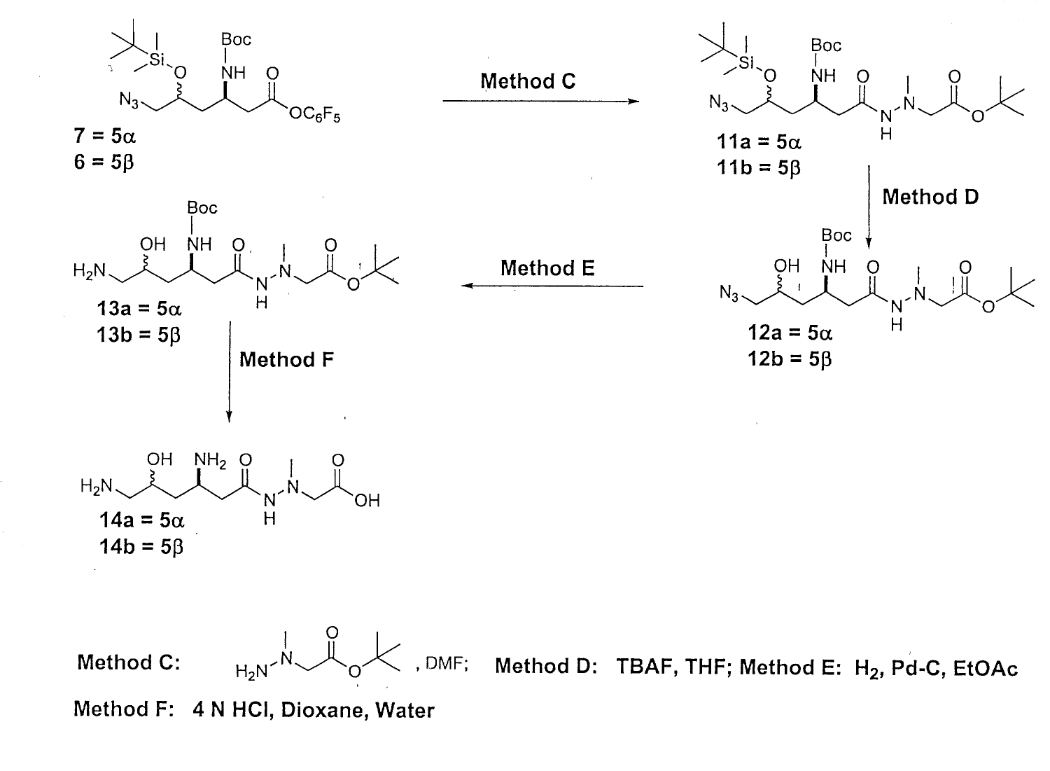

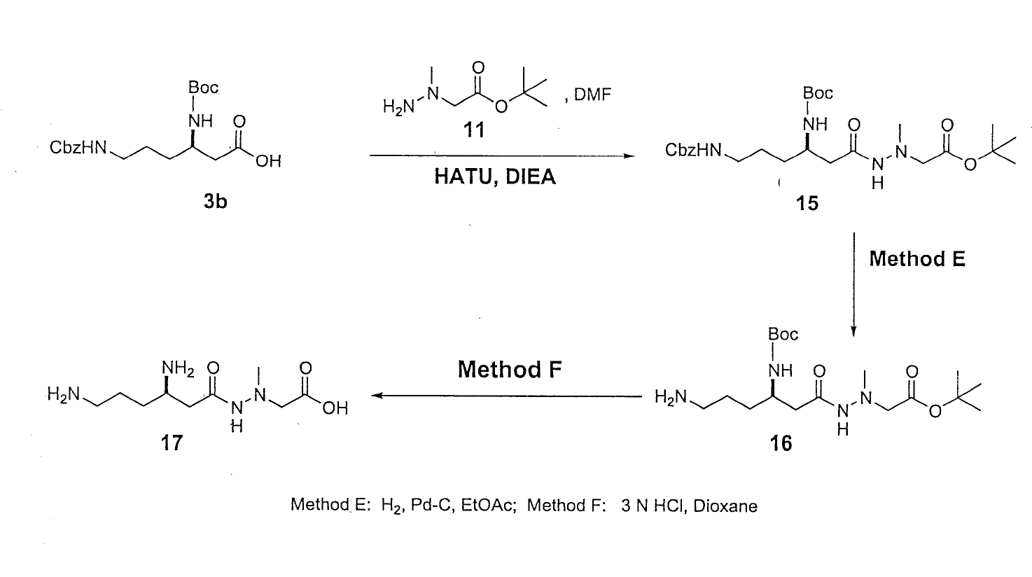

### CROSS-REFERENCE TO RELATED APPLICATIONS

This application claims the benefit of U.S. Provisional Application No. 60/308,001, filed Jul. 25, 2001, which is herein incorporated by reference in its entirety.

### BACKGROUND OF THE INVENTION

### FIELD OF THE INVENTION

The present invention is generally related to the treatment of bacterial infections in animals, preferably mammals.

### REFERENCES

The following publications, patents and patent applications are cited in this application as superscript numbers:

¹ M. Hamada et al., “A New Antibiotic, Negamycin,” J. Antibiotics, 23(3):170-71 (1970).

² Umezawa et al., “Negamycin,” U.S. Pat. No. 3,679,742, issued Jul. 25, 1972.

³ S. Kondo, et al., “Negamycin, a Novel Hydrazide Antibiotic,” J. Am. Chem. Soc., 93(23), 6305-6 (1971).

⁴ S. Shibahara et al., “The Total Synthesis of Negamycin and the Antipode,” J. Am. Chem. Soc., 94:4353-54 (1972).

⁵ Umezawa et al., “Antibiotic, Negamycin, and Processes for the Preparation Thereof,” U.S. Pat. No. 3,743,580 issued Jul. 3, 1973

⁶ W. Streicher, “Total Synthesis of Rac. Negamycin and of Negamycin Analogs,” J. Antibiotics, 31(7):725-728 (1978).

⁷ A. Pierdet et al., “Synthese Totale de la (±)-Negamycine,” Tetrahedron, 36:1763-1772 (1980).

⁸ G. Pasquet et al., “Synthesis of (±)-Negamycin and off (±)-Epinegamycin”, Tetrahedron Letters, 21:931-934 (1980).

⁹ Y.-F. Wang et al., “Stereocontrolled Synthesis of (+)-Negamycin from an Acyclic Homoallylamine by 1,3-Asymmetric Induction,” J. Am. Chem. Soc., 104:6466-6468 (1982)

¹⁰ H. Iida et al., “Enantioelective Total Synthesis of (+)-Negamycin and (−)-Epinegamycin by an Aymmetric 1,3-Dipolar Cycloaddition,” J. Am. Chem. Soc., 108:4647-4648 (1986).

¹¹ S. De Bernardo et al., “Synthesis of (+)-Negamycin from D-Glucose,” Tetrahedron Letters, 29(33):4077-4080 (1988).

¹² D. Tanner et al., “Enantioselective Total Synthesis of (+)-Negamycin,” Tetrahedron Letters, 29(19):2373-2376 (1988).

¹³ D. Tanner, “Total Synthesis of Natural Products: Some General Observations and a Specific Example. An Enantioselective Route to (+)-Negamycin,” Acta Pharm. Nord., 1(3): 145-154 (1989).

¹⁴ K. Kasahara et al., “Asymmetric Total Synthesis of (+)-Negamycin and (−)-3-Epinegamycin via Enantioselective 1,3-Dipolar Cycloaddition,” J. Org. Chem, 54:2225-33 (1989).

¹⁵ C. D. Maycock et al., “An Application of Quinic Acid to the Synthesis of Linear Homochiral Molecules: A Synthesis of (+)-Negamycin,” Tetrahedron Letters, 33(32):4633-4636 (1992).

¹⁶ J. J. Masters and L. S. Hegedus, “Palladium(II)-Assisted Difunctionalization of Monoolefins: Total Synthesis of (+)-Negamycin and (−)-5-epi-Negamycin,” J. Org. Chem., 58:4547-4554 (1993).

¹⁷ D. Socha et al., “Stereocontrolled Entry to Negamycin from D-Glucose,” Tetrahedron Letters, 36(1): 135-138 (1995).

¹⁸ S. G. Davies and O. Ichihara, “Asymmetric Synthesis of (+)-Negamycin,” Tetrahedron: Asymmetry, 7(7):1919-1922 (1996).

¹⁹ M. Shimizu et al., “Stereocontrol in the Addition of Allyl Metal Reagents to an Optically Active Imine Derived from Malic Acid, Leading to a Formal Synthesis of (+)-Negamycin,” Chemistry Letters, 467-8 (1998).

²⁰ S. Kondo et al., “Synthesis and Properties of Negamycin Analogs Modified the δ-hydroxy-β-lysine moiety,” J. Antibiotics, 29(2):208-211 (1976).

²¹ W. V. Curran, “D-3,6-Diaminohexanoic acid 2-(carboxymethyl)-2-methylhydrazide, Process of Preparing and Intermediates of Said Process,” U.S. Pat. No. 3,962,317 issued Jun. 8, 1976.

²² W. V. Curran and J. H. Boothe, “The Synthesis of Deoxynegamycin and Some Related Compounds,” J. Antibiotics, 31(9):914-918(1978).

²³ S. Mizuno et al., “Mechanism of Action of Negamycin in Escherichia Coli K12; I. Inhibition of Initiation of Protein Synthesis,” J. Antibiotics, 23(12):581-588 (1970).

²⁴ S. Mizuno et al., “Mechanism of Action of Negamycin in Escherichia Coli K12; II. Miscoding Activity in Polypeptide Synthesis Directed by Synthetic Polynucleotide,” J. Antibiotics, 23(12):589-594 (1970).

²⁵ “Negamycin Derivatives and Synthesis Thereof,” GB 1,553,416, published Sep. 26, 1979.

²⁶ Umezawa et al., “δ-Substituted Negamycin Derivatives and Syntheses,” U.S. Pat. No. 4,065,495 issued Dec. 27, 1977.

All of the above publications, patents and patent applications are herein incorporated by reference in their entirety to the same extent as if each individual publication, patent or patent application was specifically and individually to be incorporated by reference in its entirety.

### STATE OF THE ART

Negamycin is an antibiotic, isolated from the culture filtrate of three strains related to Streptomyces purpeofuscus, and was first reported by M. Hamada et al.¹ Its chemical structure is shown below:

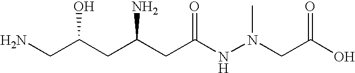

### [2-{(3R,5R)-3, 6-diamino-5-hydroxyhexanoyl}-1-methylhydrazino]acetic acid

Negamycin is produced by cultivating a strain of S. purpeofurcus in a nutrient medium under aerobic conditions, according to the protocol set forth in U.S. Pat. No. 3,679,742.² Alternatively, negamycin may also be synthesized, for example, by the reaction of (R,R)-δ-hydroxy-β-lysine and 1-methylhydrazinoacetic acid, as described by S. Kondo, et al.,³ and S. Shibahara, et al.⁴ A variety of other synthetic methods for making negamycin are also described in the art,⁵⁻⁸ including a variety of enantioselective syntheses.⁹⁻¹⁹

Deoxynegamycin is the deoxy analog of negamycin, and has the following structure:

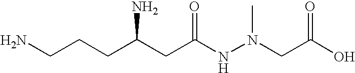

### D-3,6-diaminiohexanoic acid-2-(carboxymethyl)-2-methylhyrazide

The synthesis of deoxynegamycin, using negamycin as starting material, was first reported by S. Kondo et al.²⁰ Various other methods for the synthesis of deoxynegamycin have since been reported.²¹⁻²²

Both negamycin and deoxynegamycin display antibacterial activity. Negamycin displays low toxicity and displays strong inhibitory activity against Gram negative and Gram positive bacteria. Negamycin has been shown to inhibit initiation of protein synthesis,²³ and to induce misreading of the genetic codes of synthetic mRNAs.²⁴ Negamycin has been reported as effective for inhibiting the following organisms: Staphylococcus aureus, Escherichia coli, Shigella sonnei, Shigella flexneri, Salmonella typhosa, Salmonella typhi, Salmonella typhimurium, Salmonella enteriditis, Enterobacter sp., Klebsiella pneumoniae, Serratia marcescens, Herellea vaginicola, Proteus vulgaris, Proteus rettgeri, Pseudomonas aeruginosa, Pseudomonas fluorescens and Mycobacterium smegmatis.^{1,4,7,20,22,26}

Deoxynegamycin is reported to be useful as an antimicrobial agent, with broad-spectrum antibacterial and antifungal activity in vitro against a variety of standard laboratory microorganisms. Deoxynegamycin is reported as effective against the following organisms: Staphylococcus aureus, Escherichia coli, Shigella flexneri, Salmonella typhi, Salmonella typhimurium, Salmonella enterditis, Klebsiella pneumoniae, Enterobacter sp., Enterobacter aerogenes, Serratia marcescens, Herellea vaginicola, Proteius mirabilis, Proteus vulgris, Proteus rettgeri, Pseudomonas aeruginosa, Pseudomonas fluorescens, Mycobacterium smegmatis, Candida albicans, Cryptococcus neoformans, Trichophyton tonsurans and Trichophyton mentagraphytes.^{7,20,21,22}

It has been reported that when negamycin was kept at 37° C. for one month, its activity was reduced to 63% in aqueous solution and to 50% in 0.02N HCl aqueous solution.^{25,26} In U.S. Pat. No. 3,679,742 it is reported that after heating 5 mg/mL of negamycin solution of various pH (e.g. 1.8, 7.2, 9.2) at 60° C. for 30 minutes, 91 percent remained without decomposition.² The same patent also reports that when 2 mg/mL of negamycin solution of various pH was kept at 27° C. for a week, the following percent of negamycin remained without decomposition: 86 percent at pH 2.25, 80 percent at pH 4.90, 76 percent at pH 7.15, 89 percent at pH 9.0, and 79 percent at pH 10.2.²

Currently, peptides and other compounds having amide bonds may not be suitable for oral administration, due to the instability of such compounds to acidic conditions and/or enzymatic degradation. When this is the case, such compounds are often limited to alternative modes of administration. For example, oral administration of negamycin has not been reported, although the art describes administering negamycin by intramuscular injection, intravenous injection, interperitoneal injection, and subcutaneous injection.² In most cases, these alternative modes of administration result in greater inconvenience to the patient, as well as expense.

Thus, despite the developments reported in the art, there remains a need for effectively using negamycin and deoxynegamycin to treat bacterial infections. This invention answers this need.

### SUMMARY OF THE INVENTION

The invention relates generally to a method of treating a bacterial infection, wherein the method comprises orally administering a pharmaceutical composition to an animal (preferably a mammal), and wherein the composition comprises a pharmaceutically acceptable excipient and an antibacterial effective amount of negamycin, or a pharmaceutically acceptable salt, prodrug or isomers thereof. According to the invention, negamycin, or a pharmaceutically acceptable salt, prodrug or isomers thereof, may be orally administered in a dosage of from about 0.1 to about 100 mg/(kg animal)/day, or about 0.1 to about 50 mg/(kg animal)/day, or about 0.5 to about 20 mg/(kg animal)/day. In addition, the pharmaceutical composition may further comprise at least one additional antibiotic (preferably an antibiotic effective against gram positive bacteria).

The invention also relates to a method of treating a bacterial infection, wherein the method comprises intravenously administering a pharmaceutical composition to an animal (preferably a mammal), and wherein the composition comprises a pharmaceutically acceptable excipient and an antibacterial effective amount of deoxynegamycin, or a pharmaceutically acceptable salt, prodrug or isomers thereof. According to the invention, deoxynegamycin, or a pharmaceutically acceptable salt, prodrug or isomers thereof, may be intravenously administered in a dosage from 0.1 to about 100 mg/(kg animal)/day, or about 0.1 to about 50 mg/(kg animal)/day, or about 0.5 to about 20 mg/(kg animal)/day. In addition, the pharmaceutical composition may further comprise at least one additional antibiotic (preferably an antibiotic effective against gram positive bacteria).

The invention also relates to a method of treating a bacterial infection, wherein the method comprises administering, to an animal (preferably a mammal), an antibacterial effective amount of negamycin or deoxynegamycin, or a pharmaceutically acceptable salt, prodrug or isomers thereof, and wherein the infecting bacteria are selected from a group of bacteria consisting of the following: Acinetobacter baumanii, Citrobacter freundii, Enterobacter aerogenes, haemophilus influenzae, Moraxella catarrhalis, Staphylococcus aureus MRSA, Staphylococcus aureus GISA, Staphylococcus epidermis, Streptococcus pneumoniae PenR, Streptococcus pneumoniae PenS, Streptococcus pyogenes and Helicobacter pylori. The dosage, for example, may range from about 0.1 to about 100 mg/(kg animal)/day, or about 0.1 to about 50 mg/(kg animal)/day, or about 0.5 to about 20 mg/(kg animal)/day.

### BRIEF DESCRIPTION OF THE DRAWINGS

Schemes 1-5 show synthetic routes to both negamycin and deoxynegamycin.

### DETAILED DESCRIPTION OF THE INVENTION

As above,-this invention relates to the treatment of bacterial infections in mammals. Specifically, it relates to treatment of bacterial infections by administration of negamycin, deoxynegamycin, or a pharmaceutically acceptable salt, prodrug or isomer thereof. However, prior to describing the invention in further detail, the following terms will first be defined.

A. Definitions

“Bacterial Infection” refers to an infection resulting from the invasion of the body by bacteria, whether clinically apparent or not. “Bacteria” refers to unicellular microorganisms of the class Schizomycetes, as well as all prokaryotic organisms that are not blue-green algae. Bacteria may have a variety of shapes, for example, spheric (cocci), rod-shaped (bacilli), spiral (spirochetes), or comma-shaped (vibrio). The invasion of the body by bacteria that reproduce and multiply, may cause disease, for example, by local cellular injury, competitive metabolism, secretion of a toxin, or an antigen-antibody reaction in the host.

“Negamycin” refers to [2-{(3R,5R)-3,6-diamino-5-hydroxyhexanoyl}-1-methylhydrazino]acetic acid, and has the following chemical formula:

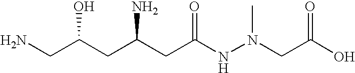

“Deoxynegamycin” refers to D-3,6-diaminiohexanoic acid-2-(carboxymethyl)-2-methylhyrazide, and has the following chemical formula:

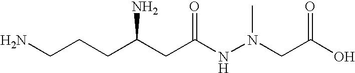

“Treating” or “treatment” of a disease includes:

(1) preventing the disease, i.e. causing the clinical symptoms of the disease not to develop in a mammal that may be exposed to or predisposed to the disease but does not yet experience or display symptoms of the disease,

(2) inhibiting the disease, i.e., arresting or reducing the development of the disease or its clinical symptoms, or

(3) relieving the disease, i.e., causing regression of the disease or its clinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

A “antibacterial effective amount” means the amount of a compound that, when administered to a mammal for treating a bacterial infection, is sufficient to effect such treatment for the infection. The “antibacterial effective amount” will vary depending on the compound, the disease and its severity and the age, weight, etc., of the mammal to be treated.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the specification and claims includes both one and more than one such excipient.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

A “pharmaceutically acceptable salt” of a compound means a salt that is pharmaceutically acceptable and that possesses the desired pharmacological activity of the parent compound. Such salts include: A “pharmaceutically acceptable salt” of a compound means a salt that is pharmaceutically acceptable and that possesses the desired pharmacological activity of the parent compound. Such salts include:

(1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-napthalenesulfonic acid, 4-toluenesulfonic acid, camphorsulfonic acid, 3-phenylpropionic acid, trimethylacetic acid, tertiary butylacetic acid, lauryl sulfuric acid, gluconic acid, glutamic acid, hydroxynapthoic acid, salicylic acid, stearic acid, muconic acid, and the like; or

(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, trimethamine, N-methylglucamine, and the like.

“Prodrug” means any compound which releases an active parent drug according to Formula (I) in vivo when such prodrug is administered to a mammalian subject. Prodrugs of a compound may be prepared by modifying functional groups present in the compound in such a way that the modifications may be cleaved in vivo to release the parent compound. Prodrugs include compounds of Formula (I) wherein a hydroxy, amino, or sulfhydryl group in compound (I) is bonded to any group that may be cleaved in vivo to regenerate the free hydroxyl, amino, or sulfhydryl group, respectively. Examples of prodrugs include, but are not limited to esters (e.g., acetate, formate, and benzoate derivatives), carbamates (e.g., N,N-dimethylamino-carbonyl) of hydroxy functional groups in compounds of Formula (I), and the like.

“Isomers” are compounds that have the same molecular formula but differ in the nature or sequence of bonding of their atoms or the arrangement of their atoms in space. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

By “antibiotic” is meant a chemical substance that has the ability to kill or inhibits the multiplication of microbes or microorganisms. Typically, an antibiotic is produced by microbes, and is harmful to other microbes. Bacteria, fungi, viruses, and protozoa are all examples of microbes. Antibiotics that are non-toxic to the host are often used as chemotherapeutic agents in the treatment of infectious diseases of man, animals, and plants.

Antibiotics exhibit activity against gram positive bacteria (gram positive agents), gram negative bacteria (gram negative agents) or are broad spectrum (i.e., agents active against both types). Examples of antibiotics that may be use include, but are not limited to, Acyclovir, Amantadine, Amikacin, Amoxicillin, Amoxicillin; Clavulanic Acid, Amphotericin B, Ampicillin, Sulbactam, Atovaquone, Azithromycin, Aztreonam, Bacitracin, Bismuth Subsalicylate, Carbenicillin, Cefaclor, Cefadroxil, Cephaloglycin, Cephaloridine, Cephalothin, Cephapirin, Cephradine, Cephahalexin, Cefamandole, Cefazolin, Cefepime, Cefixime, Cefoperazone, Cefotaxime, Cefotetan, Cefoxitin, Cefprozil, Ceftazidime, Ceftriaxone, Cefuroxime, Cephalexin,, Chloramphenicol, Chloroquine, Cidofovir, Ciprofloxacin, Clarithromycin, Clindamycin, Clofazimine, Clotrimazole, Co-trimoxazole, Cycloserine, Dapsone, Demeclocycline, Dicloxacillin, Dirithromycin, Doxycycline, Erythromycin, Ethambutol, Famciclovir, Fleroxacin, Fluconazole, Flucytosine, Foscarnet, Ganciclovir, Gentamicin, Griseofulvin, Hydroxychloroquine, Imipenem, Cilastatin, Indinavir, Interferon Alfa, Isoniazid, Itraconazole, Ketoconazole, Lamivudine, Lindane, Lomefloxacin, Loracarbef, Mebendazole, Methenamine, Methicillin, Metronidazole, Mezlocillin, Miconazole, Minocycline, Mupirocin, Nafcillin, Neomycin, Nevirapine, Nitrofurantoin, Norfloxacin, Nystatin, Ofloxacin, Oxacillin, Penicillin G, Penicillin V, Pentamidine, Piperacillin, Piperacillin; Tazobactam, Polymyxin B, Primaquine, Pyrantel, Pyrazinamide, Pyrimethamine, Quinacrine, Quinine, Ribavirin, Rifabutin, Rifampin, Rimantadine, Ritonavir, Saquinavir, Silver Sulfadiazine, Spectinomycin, Stavudine, Streptomycin, Sulfacetamide, Sulfamethoxazole, Sulfisoxazole, Terbinafine, Tetracycline, Thiabendazole, Ticarcillin, Ticarcillin; Clavulanic Acid, Tobramycin, Trimethoprim, Valacyclovir, Vancomycin, Vidarabine, Zalcitabine, Zidovudine, and Zyvox. Examples of gram positive agents include, but are not limited to, Zyvox and Cephalosporins (e.g., Cefadroxil, Cefazolin, Cephalexin, Cephaloridine, Cephalothin, Cephapirin and Cephradine). Examples of gram negative agents include, but are not limited to, Aminoglycosides (e.g., Streptomycin, Gentamycin, Sisomicin, Netilmicin, Amikacin, Neomycin, Kanamycin and Tobramycin.). Examples of broad spectrum agents include, but are not limited to, Penicillins (e.g., Penicillin G, Ampicillin, Amoxicillin, Carbenicillin, Ticarcillin, Azlocillin and Mezlocillin), third generation Cephalosporins (e.g., Cefdinir, Cefixime, Cefoperazone, Cefotaxime, Cefpodoxime, Ceftazidime, Ceftibuten, Ceftizoxime and Ceftriaxone), Quinolones (e.g., Gatifloxacin, Grepafloxacin, Sparfloxacin, Tosufloxacin, Clinafloxacin, Moxifloxacin and Trovafloxacin) and Macrolides (e.g., Erythromycin, Dirithromycin and Clarithromycin).

B. Method of Obtaining Negamycin

Negamycin is obtained either through laboratory synthesis or isolation from natural sources.¹⁻⁹ Schemes 1-4 and the Examples below describe one synthetic route to the compound. In reference to the schemes, acid 1a is converted to homologated ester 2a, which is hydrolyzed to 3a. Acid 3a is cyclized upon treatment with I₂, and the resulting primary iodide is displaced with azide to form 4. Hydrolysis of the lactone (4), protection of the formed secondary alcohol with TBDMS-Cl and esterification affords pentafluorophenyl ester 5. Diastereomers 6 and 7 are separated by column chromatography, with 7 being taken forward in the negamycin synthesis. Compound 7 is reacted with hydrazine 10 to afford hydrazide 11a, which is deprotected (12a), reduced (13a) and finally deprotected to provide negamycin (14a).

C. Method of Obtaining Deoxynegamycin

Deoxynegamycin is obtained through laboratory synthesis.²² Schemes 1 and 5 and the Examples below describe one synthetic route to the compound. In reference to the schemes, acid 1b is converted to homologated ester 2b, which is hydrolyzed to 3b. Reaction of acid 3b with hydrazine 11 provides hydrazide 15. Deprotection of 15 and the resulting primary amine (16) on acidic treatment affords deoxynegamycin (17).

D. Formulations

When employed as pharmaceuticals, negamycin or deoxynegamycin, as well as their pharmaceutically acceptable salts, prodrugs or isomers, are usually administered in the form of pharmaceutical compositions. These compounds can be administered by a variety of routes including oral, rectal, transdermal, subcutaneous, intravenous, intramuscular, and intranasal. These compounds are effective as both injectable, topical, and oral compositions. Such compositions are prepared in a manner well known in the pharmaceutical art.

This invention includes pharmaceutical compositions which contain negamycin or deoxynegamycin, or a pharmaceutically acceptable salt, prodrug or isomer thereof, as the active ingredient, associated with pharmaceutically acceptable carriers. In making the compositions of this invention, the active ingredient is usually mixed with an excipient, diluted by an excipient or enclosed within such a carrier which can be in the form of a capsule, sachet, paper or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions are preferably formulated in a unit dosage form, each dosage containing from about 5 to about 100 mg, more usually about 10 to about 30 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Preferably, the compound of formula I above is employed at no more than about 20 weight percent of the pharmaceutical composition, more preferably no more than about 15 weight percent, with the balance being pharmaceutically inert carrier(s).

The active compound is effective over a wide dosage range and is generally administered in a pharmaceutically effective amount. It will be understood, however, that the amount of the compound actually administered will be determined by a physician, in the light of the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices which deliver the formulation in an appropriate manner.

The topical formulations of the present invention can be presented as, for instance, ointments, creams or lotions, solutions, salves, emulsions, plasters, eye ointments and eye or ear drops, impregnated dressings and aerosols, and can contain appropriate conventional additives such as preservatives, solvents to assist drug penetration and emollients in ointments and creams.

The formulations can also contain compatible conventional carriers, such as cream or ointment bases and ethanol or oleyl alcohol for lotions. Such carriers can be present, for example, from about 1% up to about 99% of the formulation. For example, they can form up to about 80% of the formulation.

Tablets and capsules for oral administration can be in unit dose presentation form, and can contain conventional excipients such as binding agents, for example syrup, acacia, gelatin, sorbitol, tragacanth, or polyvinylpyrollidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets can be coated according to methods well known in standard pharmaceutical practice.

Oral liquid preparations can be in the form of, for example, aqueous or oily suspensions, solutions, emulsions, syrups or elixirs, or can be presented as a dry product for reconstitution with water or another suitable vehicle before use. Such liquid preparations can contain conventional additives, such as suspending agents, for example sorbitol, methyl cellulose, glucose syrup, gelatin, hydroxyethyl cellulose, carboxymethyl cellulose, aluminium stearate gel or hydrogenated edible fats; emulsifying agents, for example lecithin, sorbitan monooleate, or acacia; non-aqueous vehicles (which can include edible oils), for example almond oil, oily esters such as glycerine, propylene glycol, or ethyl alcohol; preservatives, for example methyl or propyl p-hydroxybenzoate or sorbic acid, and, if desired, conventional flavoring or coloring agents.

For parenteral administration, fluid unit dosage forms are prepared utilizing the compound and a sterile vehicle, water being preferred. The compound, depending on the vehicle and concentration used, can be either suspended or dissolved in the vehicle or other suitable solvent. In preparing solutions, the compound can be dissolved in water for injection and filter sterilized before filling into a suitable vial or ampoule and sealing. Advantageously, agents such as a local anesthetic preservative and buffering agents can be dissolved in the vehicle. To enhance the stability, the composition can be frozen after filling into the vial and the water removed under vacuum. The dry lyophilized powder is then sealed in the vial and an accompanying vial of water for injection can be supplied to reconstitute the liquid prior to use. Parenteral suspensions are prepared in substantially the same manner except that the compound is suspended in the vehicle instead of being dissolved and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

The compositions can contain, for example, from about 0.1% by weight to about 99% by weight, e.g., from about 10-60% by weight, of the active material, depending on the method of administration. Where the compositions comprise dosage units, each unit will contain, for example, from about 1-500 mg of the active ingredient. The dosage as employed for adult human treatment will range, for example, from about 1 to 3000 mg per day, for instance 1500 mg per day depending on the route and frequency of administration. Such a dosage corresponds to about 0.015 to 50 mg/kg per day. Suitably the dosage is, for example, from about 5 to 20 mg/kg per day.

The following formulation examples illustrate representative pharmaceutical compositions of the present invention.

### Formulation Example 1

Hard gelatin capsules containing the following ingredients are prepared:

| | Quantity |
|---|---|
| Ingredient | (mg/capsule) |
| Active Ingredient | 30.0 |
| Starch | 305.0 |
| Magnesium stearate | 5.0 |

The above ingredients are mixed and filled into hard gelatin capsules in 340 mg quantities.

### Formulation Example 2

A tablet formula is prepared using the ingredients below:

| | Quantity |
|---|---|
| Ingredient | (mg/tablet) |
| Active Ingredient | 25.0 |
| Cellulose, microcrystalline | 200.0 |
| Colloidal silicon dioxide | 10.0 |
| Stearic acid | 5.0 |

The components are blended and compressed to form tablets, each weighing 240 mg.

### Formulation Example 3

A dry powder inhaler formulation is prepared containing the following components:

| Ingredient | Weight % |
|---|---|
| Active Ingredient | 5 |
| Lactose | 95 |

The active ingredient is mixed with the lactose and the mixture is added to a dry powder inhaling appliance.

### Formulation Example 4

Tablets, each containing 30 mg of active ingredient, are prepared as follows:

| | Quantity |
|---|---|
| Ingredient | (mg/tablet) |
| Active Ingredient | 30.0 mg |
| Starch | 45.0 mg |
| Microcrystalline cellulose | 35.0 mg |
| Polyvinylpyrrolidone | 4.0 mg |
| (as 10% solution in sterile water) | |
| Sodium carboxymethyl starch | 4.5 mg |
| Magnesium stearate | 0.5 mg |
| Talc | 1.0 mg |
| Total | 120 mg |

The active ingredient, starch and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50° to 60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 120 mg.

### Formulation Example 5

Capsules, each containing 40 mg of medicament are made as follows:

| | Quantity |
|---|---|
| Ingredient | (mg/capsule) |
| Active Ingredient | 40.0 mg |
| Starch | 109.0 mg |
| Magnesium stearate | 1.0 mg |
| Total | 150.0 mg |

The active ingredient, starch, and magnesium stearate are blended, passed through a No. 20 mesh U.S. sieve, and filled into hard gelatin capsules in 150 mg quantities.

### Formulation Example 6

Suppositories, each containing 25 mg of active ingredient are made as follows:

| Ingredient | Amount |
|---|---|
| Active Ingredient | 25 mg |
| Saturated fatty acid glycerides to | 2,000 mg |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

### Formulation Example 7

Suspensions, each containing 50 mg of medicament per 5.0 mL dose are made as follows:

| Ingredient | Amount |
|---|---|
| Active Ingredient | 50.0 mg |
| Xanthan gum | 4.0 mg |
| Sodium carboxymethyl cellulose (11%) | 50.0 mg |
| Microcrystalline cellulose (89%) | |
| Sucrose | 1.75 g |
| Sodium benzoate | 10.0 mg |
| Flavor and Color | q.v. |
| Purified water to | 5.0 mL |

The active ingredient, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

### Formulation Example 8

| | Quantity |
|---|---|
| Ingredient | (mg/capsule) |
| Active Ingredient | 15.0 mg |
| Starch | 407.0 mg |
| Magnesium stearate | 3.0 mg |
| Total | 425.0 mg |

The active ingredient, starch, and magnesium stearate are blended, passed through a No. 20 mesh U.S. sieve, and filled into hard gelatin capsules in 425.0 mg quantities.

### Formulation Example 9

A subcutaneous formulation may be prepared as follows:

| Ingredient | Quantity |
|---|---|
| Active Ingredient | 5.0 mg |
| Corn Oil | 1.0 mL |

### Formulation Example 10

A topical formulation may be prepared as follows:

| Ingredient | Quantity | |
|---|---|---|
| Active Ingredient | 1-10 | g |
| Emulsifying Wax | 30 | g |
| Liquid Paraffin | 20 | g |
| White Soft Paraffin | to 100 | g |

The white soft paraffin is heated until molten. The liquid paraffin and emulsifying wax are incorporated and stirred until dissolved. The active ingredient is added and stirring is continued until dispersed. The mixture is then cooled until solid.

Another preferred formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252, issued Jun. 11, 1991, herein incorporated by reference. Such patches may be constructed for continuous, pulsatile, or on demand delivery of pharmaceutical agents.

Frequently, it will be desirable or necessary to introduce the pharmaceutical composition to the brain, either directly or indirectly. Direct techniques usually involve placement of a drug delivery catheter into the host's ventricular system to bypass the blood-brain barrier. One such implantable delivery system used for the transport of biological factors to specific anatomical regions of the body is described in U.S. Pat. No. 5,011,472 which is herein incorporated by reference.

Indirect techniques, which are generally preferred, usually involve formulating the compositions to provide for drug latentiation by the conversion of hydrophilic drugs into lipid-soluble drugs. Latentiation is generally achieved through blocking of the hydroxy, carbonyl, sulfate, and primary amine groups present on the drug to render the drug more lipid soluble and amenable to transportation across the blood-brain barrier. Alternatively, the delivery of hydrophilic drugs may be enhanced by intra-arterial infusion of hypertonic solutions which can transiently open the blood-brain barrier.

Other suitable formulations for use in the present invention can be found in Remington's Pharmaceutical Sciences, Mace Publishing Company, Philadelphia, Pa., 17th ed. (1985), which is hereby incorporated in its entirety.

E. Utility

Negamycin or deoxynegamycin, or a pharmaceutically acceptable salt, prodrug or isomer thereof is useful in treating bacterial infections in animals, and preferably in mammals including humans.

As noted above, the compounds described herein are suitable for use in a variety of drug delivery systems described above. Additionally, in order to enhance the in vivo serum half-life of the administered compound, the compounds may be encapsulated, introduced into the lumen of liposomes, prepared as a colloid, or other conventional techniques may be employed which provide an extended serum half-life of the compounds. A variety of methods are available for preparing liposomes, as described in, e.g., Szoka, et al., U.S. Pat. Nos. 4,235,871, 4,501,728 and 4,837,028, each of which is incorporated herein by reference.

The amount of compound administered to the patient will vary depending upon what is being administered, the purpose of the administration, such as prophylaxis or therapy, the state of the patient, the manner of administration, and the like. In therapeutic applications, compositions are administered to a patient already suffering from a bacterial infection in an amount sufficient to at least partially arrest further onset of the symptoms of the disease and its complications. An amount adequate to accomplish this is defined as “therapeutically effective dose.” Amounts effective for this use will depend on the judgment of the attending clinician depending upon factors such as the degree or severity of the bacterial infection in the patient, the age, weight and general condition of the patient, and the like. Preferably, for use as therapeutics, the compounds described herein are administered at dosages ranging from about 0.1 to about 500 mg/kg/day.

In prophylactic applications, compositions are administered to a patient at risk of developing a bacterial infection in an amount sufficient to inhibit the onset of symptoms of the disease. An amount adequate to accomplish this is defined as “prophylactically effective dose.” Amounts effective for this use will depend on the judgment of the attending clinician depending upon factors such as the age, weight and general condition of the patient, and the like. Preferably, for use as prophylactics, the compounds described herein are administered at dosages ranging from about 0. 1 to about 500 mg/kg/day.

As noted above, the compounds administered to a patient are in the form of pharmaceutical compositions described above. These compositions may be sterilized by conventional sterilization techniques, or may be sterile filtered. When aqueous solutions are employed, these may be packaged for use as is, or lyophilized, the lyophilized preparation being combined with a sterile aqueous carrier prior to administration. The pH of the compound preparations typically will be between 3 and 11, more preferably from 5-9 and most preferably from 7 and 8. It will be understood that use of certain of the foregoing excipients, carriers, or stabilizers will result in the formation of pharmaceutical salts.

The following synthetic and biological examples are offered to illustrate this invention and are not to be construed in any way as limiting the scope of this invention. Unless otherwise stated, all temperatures are in degrees Celsius.

### EXAMPLES

The abbreviations in the examples below have their generally acceptable meaning.

In the examples below, all temperatures are in degrees Celsius (unless otherwise indicated) and the following procedures were used to prepare the compounds as indicated.

A. Preparation of Compounds

### General Methods

Method A: To a solution of amino acid (40 mmol) in anhydrous tetrahydrofuran (120 mL) at −15° C. was added N-methylmorpholine followed by dropwise addition of isobutyl chloroformate (5.7 mL, 44 mmol). The reaction mixture was stirred at −15° C. for 30 min and then it was brought to 0° C. To this was added ethereal solution of diazomethane (prepared from diazald 42.8 g, 200 mmol). The resultant reaction mixture was stirred at room temperature for 4 h. Excess diazomethane was decomposed by dropwise addition of acetic acid and diluted the reaction mixture with ether (400 mL). This was washed with brine (2×100 mL), saturated aqueous sodium bicarbonate (2×100 mL), saturated aqueous ammonium chloride (2×100 mL), and finally with brine (100 mL). The organic layer was dried (MgSO₄) and the residue obtained, upon removal of solvent under reduced pressure, was used immediately in the next step without further purification.

To a stirred solution of diazoketone, prepared above, in anhydrous methanol (200 mL round bottom flask covered with aluminum foil) was added a solution of silver benzoate (600 mg) in triethylamine (6 mL, exothermic reaction) and continued stirring at room temperature overnight. The brown solid was filtered through a pad of celite and the filtrate was concentrated in vacuo. The resultant residue was dissolved in ether (400 mL), washed with water (3×100 mL), dried the organic layer (MgSO₄), and then concentrated in vacuo to give the product.

Method B: To a solution of methyl ester (15.20 mmol) in methanol (30 ml) at 0° C. was added 1 N lithium hydroxide solution (35 mL). The reaction mixture was stirred at room temperature for 14 h, removed methanol in vacuo, and then the residue was dissolved in water (50 mL). This was extracted with ether (2×50 mL) to remove neutral impurities. Acidified the aqueous layer with 1 N hydrochloric acid at 0° C. and extracted with 1:1 mixture of ether/ethyl acetate (3×75 mL), after saturating the aqueous layer with sodium chloride. The combined organic layer was dried (MgSO₄) and then removal of the solvent in vacuo gave the desired acid.

Method C: To a solution of pentafluorophenyl ester (0.88 mmol) in anhydrous N,N-dimethylformamide (1 mL) was added hydrazine derivative 10 (200 mg, 1.42 mmol) and the mixture was stirred at room temperature for 3 h. Removed the solvent in vacuo, the residue was taken in ether (75 mL), and washed with 5% sodium hydroxide solution (2×20 mL). The organic layer was dried (MgSO₄), concentrated in vacuo, and the resultant residue was purified by column chromatography to get the hydrazide.

Method D: To a stirred solution of silyloxy ether (3.31 mmol) in tetrahydrofuran (10 mL) was added 1 M solution of TBAF in tetrahydrofuran (3.63 mL, 3.63 mmol). The reaction mixture was stirred at room temperature for 75 min and then diluted with ether (150 mL). This was washed with brine (2×50 mL), water (2×50 mL), dried the organic phase (MgSO₄), and then concentrated in vacuo. The residue was purified by column chromatography to get hydroxy derivative.

Method E: To a stirred solution of azide (0.23 mmol) in ethyl acetate (1.8 mL) was added 2 drops of acetic acid and then Pd-C (15 mg, 5%). This was subjected to catalytic hydrogenation using a balloon full of hydrogen at room temperature for 3 h. The catalyst was filtered through a pad of celite and was washed with ethyl acetate (10 mL). The combined filtrate was concentrated in vacuo and the resultant residue was purified by ion exchange column (Mega bound elut SCX column, sulfonic acid bound to silica gel). This column was eluted with methanol to remove non-basic impurities and then eluted with ethanolic ammonia (1 M solution) to get the desired amine. Solvent was removed to get the residue and the residue was taken in water (3 mL) and lyophilized to give the amine.

Method F: The protected amine (0.198 mmol) was taken in 4 N hydrochloric acid in dioxane (1.5 mL) and then added 3 drops of water (slightly exothermic). The resultant solution stirred at room temperature for 5 h and then removed solvent in vacuo. The residue was dissolved in water (5 mL), extracted with ether (2×5 mL), and lyophilized to get the amine hydrochloride.

### Compound Synthesis

### Example 1

Step 1: The ester 2a was prepared according to Method A and the resultant residue was purified by silica gel column chromatography using a mixture of 9:1 hexanes in ethyl acetate as an eluent to get methyl (3R)-t-butoxycarbonylaminohex-5-enoate 2a in 76% yield. R_{f}=0.13 (9:1 hexanes/ethyl acetate, silica gel). ¹H NMR (300 MHz, CDCl₃) δ1.42 (s, 9H), 2.30 (bt, J=6.6 Hz, 2H), 2.52 (d, J=5.4 Hz, 2H), 3.68 (s, 3H), 4.08 (bs, 1H), 4.94 (bs, 1H), 5.01 (m, 2H), 5.80 (m, 1H). MS (m/z): 244 (M+H).

Step 2: (3R)-t-Butoxycarbonylaminohex-5-enoic acid 3a was prepared from the corresponding ester 2a according to the Method B in quantitative yield. R_{f}=0.17 (5% methanol in dichloromethane, silica gel). ¹H NMR (300 MHz, CDCl₃) δ1.44 (s, 9H), 2.35 (bt, J=7.2 Hz, 2H), 2.59 (bs, 2H), 3.99 (bs, 1H), 4.95 (bs, 1H), 5.13 (m, 2H), 5.78 (m, 1H). MS (m/z): 252 (M+Na).

Step 3: To a stirred solution of sodium bicarbonate (15 g, 17.65 mmol) in water (125 mL) was added (3R)-t-butoxycarbonylaminohex-5-enoic acid 3a (6.0 g, 26.16 mmol) followed by dichloromethane (200 mL). This was cooled to 0° C. and added potassium iodide (15 g) solution in water (20 mL) followed by solid iodine (12.5 g, 49.40 mmol). After completion of addition, the cold bath was removed and the reaction mixture was continuously stirred at room temperature for 2.5 h. The reaction mixture was diluted with dichloromethane (300 mL), the organic layer was separated, and the aqueous layer was extracted with dichloromethane (100 mL). The combined organic layer was washed with dilute solution of potassium hydrogensulfate, dried (MgSO₄), and removed solvent in vacuo. The residue thus obtained was used in the next step immediately. R_{f}=0.25 (1:1 hexanes/ethyl acetate, silica gel).

Step 4: Product from the above step was dissolved in anhydrous N,N-dimethylformamide (50 mL) and to this was added sodium azide (5 g). The reaction mixture was kept stirring at 60° C. for 10 h. Removed N,N-dimethylformamide under reduced pressure and the residue was dissolved in 1:1 mixture of ethyl acetate and ether (300 mL). This was washed with water (3×100 mL), the organic layer was dried (MgSO₄) and concentrated in vacuo. The residue was purified by column chromatography using hexanes/EtOAc (3:2) as an eluent to get (3R)-t-butoxycarbonylamino-(5R/S)-azidomethyl-δ-lactone 4 (4.5 g, 62%). R_{f}=0.19 (1:1 hexanes/ethyl acetate, silica gel). ¹H NMR (300 MHz, CDCl₃) δ1.44 (s, 9H), 1.93-2.41 (m, 2H), 2.57-3.01 (m, 2H), 3.41-3.61 (m, 2H), 4.10-4.15 (m, 1H), 4.38-4.56 (m, 1H) 4.64-4.72 (m, 1H). ¹³C NMR (75 MHz, CDCl₃) δ23.76, 25.94, 27.74, 31.02, 32.04, 38.12, 38.91, 49.41, 49.61, 70.49, 71.93, 75.43, 150.40, 150.65, 164.79. MS(m/z): 270 (M), 293 (M+Na).

Step 5: To a solution of (3R)-t-butoxycarbonylamino-(SR/S)-azidomethyl-δ-lactone 4 (3.7 g, 14 mmol) in methanol (50 mL) at 0° C. was added lithium hydroxide solution (0.7 g in 5 nL of water). The reaction mixture was stirred at room temperature for 2 h and methanol was removed in vacuo. The residue was dissolved in water (50 mL), and then extracted with ether (2×50 mL) to remove neutral impurities. The aqueous layer was neutralized with 1 N hydrochloric acid and then extracted with 1:1 mixture of ether/ethyl acetate (3×100 mL). The combined organic layer was dried (MgSO₄) followed by removal of solvent in vacuo to give the residue (hydroxy acid), which was used in the next step without any further purification. MS (m/z): 289 (M+H).

Step 6: To a solution of hydroxy acid, prepared above, in anhydrous dichloromethane (100 mL) was added imidazole (4 g, 58.82 mmol). This was cooled to 0° C. and then added t-butyldimethylsilyl chloride (4.5 g, 29.85 mmol). The reaction mixture was stirred at 0° C. for 20 min and then at room temperature for 18 h. The mixture was diluted with dichloromethane (150 mL) and washed with water (2×100 mL). The organic layer was dried (MgSO₄) and then removed the solvent in vacuo to get silylated derivative which was used in the next step without any further purification.

Step 7: To a solution of silyl ester, prepared above, in methanol (50 mL) was added potassium carbonate (1.93 g, 13.86 mmol) dissolved in water (20 mL). The reaction mixture was stirred at room temperature for 2 h and then removed methanol in vacuo. The residue was diluted with water (100 mL) and was extracted with ether. The ether layer was discarded while the aqueous layer was neutralized with 0.5 N hydrochloric acid at 0° C. This was extracted with 1:1 mixture of ethyl acetate and ether (3×100 mL). The combined organic layer was dried (MgSO₄) and then removal of solvent in vacuo to give (3R)-t-butoxycarbonylamino-5-(R/S)-t-butyldimethylsilyloxy-6-azidohexanoic acid (4.5 g, 80%). R_{f}=0.2 (5% methanol in dichloromethane, silica gel). ¹H NMR (300 MHz, CDCl₃) δ0.0 (s, 3H), 0.02 (s, 3H), 0.82 (s, 9H), 1.35 (s, 9H), 1.71 (bs, 2H), 2.54 (bs, 2H), 3.12-3.40 (m, 2H), 3.81 (bm, 2H), 4.98 (bs, 1H). MS (m/z): 403 (M+H).

Step 8: To a stirred solution of (3R)-t-butoxycarbonylamino-(5R/S)-t-butyldimethylsilyloxy-6-azidohexanoic acid (1.8 g, 4.47 mmol) in anhydrous dichloromethane (20 mL) and pyridine (5 mL) at 0° C. was added pentafluorophenyl trifluoroacetate (0.92 mL). The resultant reaction mixture was stirred at room temperature for 24 h and then diluted with dichloromethane (100 mL). This was washed with ice cold 0.3 N hydrochloric acid (3×75 mL), ice cold 0.1 N sodium hydroxide solution (3×50 mL). The organic layer was dried (MgSO₄), and concentrated in vacuo to get pentfluorophenyl (3R)-t-butoxycarbonylamino-(5R/S)-t-butyldimethylsilyloxy-6-azidohexonate 5, which was purified by column chromatography using 10:1 hexane/EtOAc to get pure product (2.01 g, 79%, diastereomeric mixture). The diastreoisomers (6:7, approximately 1:1 ratio) were separated by column chromatography using 10:1 hexane/EtOAc. Isomer A (6, HR_{f}) R_{f}=0.16 (9:1 hexanes/ehthyl acetate, silica gel, plate was developed two times). ¹H NMR (300 MHz, CDCl₃) δ0.01 (s, 3H), 0.02, (s, 3H), 0.89 (s, 9H), 1.44 (s, 9H), 1.73-1.94 (m, 2H), 2.8-2.93 (m, 2H), 3.08-3.34 (m, 2H), 3.78-3.96 (m, 2H), 4.83-4.98 (m, 1H). MS (m/z): 569 (M+H). Isomer B (7, LR_{f}) R_{f}=0.14 (9:1 hexanes/ethyl acetate, silica gel, plate was developed two times). ¹H NMR (300 MHz, CDCl₃) δ0.01 (s, 3H), 0.02, (s, 3H), 0.81 (s, 9H), 1.33 (s, 9H), 1.73 (bm, 2H), 2.78-2.98 (m, 2H), 3.07-3.28 (m, 2H), 3.84-3.87 (m, 1H), 3.83 (m, 1H), 3.98((bm, 1H), 4.85 (bd, 1H). MS (m/z): 569 (M+H).

Step 9: To a stirred solution of N-methylhydrazine (3.45 mL, 75 mmol) and triethylamine (14.8 mL) in dichloromethane (250 mL) at 0° C. was added a solution of t-butyl-2-bromoacetate (11.07 g, 75 mmol) in dichloromethane (125 mL) over a period of 30 min. The reaction mixture was continuously stirred at 0° C. for additional 2 h and then at ambient temperature for 14 h. This was concentrated in vacuo, the precipitated salt was filtered off, and washed with ether. The combined filtrate was concentrated in vacuo to get the crude product which was purified by flash column chromatography using ethyl acetate and methanol mixture (95:5 ) to afford the hydrazine 10 (9.5 g, 79%). R_{f}=0.3 (5% methanol in ethyl acetate). ¹H NMR (300 MHz, CDCl₃) δ1.48 (s, 9H), 2.62 (s, 3H), 3.32 (s, 2H). MS (m/z): 161 (M+H).

Step 10: The hydrazide 11a was prepared using pentafluorophenyl ester 7 and hydrazine 10 according to the Method C in 94% yield, after purification by column chromatography using 5% methanol in dichloromethane. R_{f}=0.37 (5% methanol in dichloromethane, silica gel). ¹H NMR (300 MHz, CDCl₃) δ0.02 (s, 6H), 0.80 (s, 9H), 1.36 (s, 9H), 1.37 (s, 9H), 1.59-1.73 (m, 2H), 2.17-2.24 (m, 2H), 2.62 (s, 3H), 3.02-3.40 (m, 2H), 3.45 (s, 2H), 3.83 (m, 2H), 5.13-5.19 (m, 1H) 7.20 (bs, 1H). MS (m/z): 545 (M+H).

Step 11: The hydroxy derivative 12a was prepared from silyl ether 11a according to the Method D in 70% yield, after purification by column chromatography using 10% methanol in dichloromethane. R_{f}=0.15 (5% methanol in dichloromethane, silica gel, plate was developed two times). ¹H NMR (300 MHz, CDCl₃) δ1.42 (s, 9H), 1.46 (s, 9H), 1.53-1.85 (m, 2H), 2.17-3.01 (m, 2H), 2.73 (s, 3H), 3.24 (d, J=2.4 Hz, 2H), 3.38-3.5 (m, 2H), 3.80 (m, 1H), 4.06-4.16 (m, 1H), 4.05 (m, 1H), 5.97-6.06 (dd, J=9Hz, 1H) 7.26 & 7.32 (two s, 1H). MS (m/z): 431 (M+H).

Step 12: The amine 13a was prepared from the azide 12a following the general Method E in 84% yield. ¹H NMR (300 MHz, CDCl₃) δ1.43 (s, 9H), 1.47 (s, 9H), 1.48-1.63 (m, 2H), 2.25-2.75 (m, 4H), 2.71 & 2.75 (two s, 3H), 3.39-3.55 (m, 4H), 4.10-4.18 (m, 2H), 5.92-6.01 (m, 1H), 7.26 & 7.4 (two s, 1H), 7.95 (s, 1H). MS (m/z): 405 (M+H).

Step 13: Negamycin 14 was prepared from amine 13a in 86% yield according to the Method F. ¹H NMR (300 MHz, D₂0) δ1.58-1.81 (m, 2H), 2.47-2.57 (m 2H), 2.53 (s, 3H), 2.80-3.01 (m, 2H), 3.51 (s, 2H), 3.52-3.76 (m, 2H), 3.86-3.98 (m, 1H). MS (m/z): 249 (M+H).

### Example 2

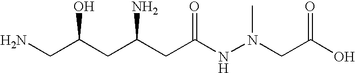

Step 1: The hydrazide 11b was prepared from pentafluorophenyl ester 6 and hydrazine derivative 10 according to the Method C in 96% yield, after purification by column chromatography using 5% methanol in dichloromethane. R_{f}=0.43 (5% methanol in dichloromethane, silica gel). ¹H NMR (300 MHz, CDCl₃) δ0.00 (s, 6H), 0.82 (s, 9H), 1.39 (s, 9H), 1.40 (s, 9H), 1.71-1.73 (m, 2H), 2.06-2.42 (m, 2H), 2.64 & 2.66 (two s, 3H), 3.11-3.42 (m, 2H), 3.46 (s, 2H), 3.60-3.94 (m, 2H), 5.40 (bs, 1H), 7,26 (bd, 1H). MS (m/z): 545 (M+H).

Step 2: The hydroxy derivative 12b was prepared from silyl ether 11b according to the Method D in 70% yield, after purification by column chromatography using 10% methanol in dichloromethane. R_{f}=0.15 (5% methanol in dichloromethane, plate was developed two times). ¹H NMR (300 MHz, CDCl₃) δ1.39 (s, 9H), 1.44 (s, 9H), 1.72-1.78 (m, 2H), 2.31-3.0 (m, 2H), 2.67 & 2.71 (two s, 3H), 3.29 (d, J=2.4 Hz, 2H), 3.30-3.53 (m, 2H), 3.60-4.15 (m, 2H), 5.64 (m, 1H), 7.40 & 7.46 (two s, 1H). MS (m/z): 431 (M+H).

Step 3: The amine 13b was prepared from the azide 12b following the general Method E in 92% yield. ¹H NMR (300 MHz, CDCl₃) δ1.42 (s, 9H), 1.47 (s, 9H), 1.63-1.70 (m, 2H), 2.39-2.87 (m, 7H), 3.40-3.63 (m, 4H), 3.98-4.06 (m, 2H), 5.57 (bs, 1H), 7.26 (bs, 1H), 8.04 (s, 1H). MS (m/z): 405 (M+H).

Step 4: 5-Epinegamycin 14b was synthesized from the amine 13b according to Method F in 87% yield. ¹H NMR (300 MHz, D₂0) δ1.59 (m, 2H), 2.36-2.58 (m, 2H), 2.53 (s, 3H), 2.74-3.00 (m, 2H), 3.46-3.58 (m, 1H), 3.51 (s, 2H), 3.62-3.68 (m, 1H), 3.83-3.91 (m, 1H). MS (m/z): 249 (M+H).

### Example 3

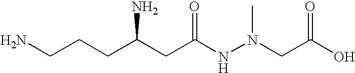

Step 1: The ester 2b was prepared according to Method A and the resultant residue was purified by column chromatography using 1:1 hexanes/ethyl acetate mixture as an eluent to get methyl (3R)-t-butoxycarbonylamino-6-benzyloxycarbonylaminohexanate 2b in 93% yield. R_{f}=0.29 (1:1 hexanes/ethyl acetate, silica gel). ¹H NMR (300 MHz, CDCl₃) δ1.63 (s, 9H), 1.72 (bm, 4H), 2.70 (m, 2H), 3.41 (m, 2H), 3.88 (s, 3H), 4.20 (bs, 1H), 5.15 (bs, 1H), 5.18 bs, 1H), 5.25 (s, 2H), 7.47 (m, 5H). MS (m/z): 295 (M+H-Boc).

Step 2: (3R)-t-Butoxycarbonylamino-6-benzyloxycarbonylaminohexnoic acid 3b was prepared from the corresponding ester 2b according to the Method B in 87% yield. Rₜ=4.61 min (50×4.6 mm, C18 column, 5% Acetonitrile in water to 100% acetonitrile over 10 min, linear gradient). ¹H NMR (300 MHz, CDCl₃). δ1.62 (s, 9H), 1.75 (bs, 4H), 2.70 (bs, 2H), 3.40 (bs, 2H), 4.10 (bs, 1H), 5.28 (s, 2H), 7.46 (s, 5H). MS (m/z): 281 (M+H-Boc).

Step 3: To a stirred mixture of β-amino acid 3b (1.5 g, 3.94 mmol), hydrazine 10 (0.758 g, 1.2 mmol) and HATU (1.79 g, 4.73 mmol) in anhydrous N,N-dimethylformamide (10 mL) at 0° C. was added diisopropylethylamine (2.47 mL, 14.2 mmol) and continued stirring at 0° C. for 1 h. After stirring the reaction mixture overnight at ambient temperature, DMF was removed under reduced pressure. The residue was taken in ether (300 mL), washed with water (100 mL), dried the organic layer (MgSO₄), and then removal of solvent in vacuo gave the product which was purified by column chromatography using ethyl acetate as an eluent to give the hydrazide 15 (1.6 g, 77%). R_{f}=0.34 (95:1 Ethyl acetate/methanol, silica gel). MS (m/z): 545.8 (M+Na).

Step 4: The amine hydrazide 16 was prepared from the derivative 15 using Method E, without acetic acid, in 65% yield. MS (m/z): 411.7 (M+Na).

Step 5: Deoxynegamycin 17 was prepared from the amine 16 using Method F in 76% yield. This was purified by ion exchange column (CG50, 1% ammonium hydroxide as an eluent) ¹H NMR (300 MHz, D₂0) δ1.45 (bs, 4H), 2.30-2.40 (m, 2H), 2.50 (s, 3H), 2.80 (bs, 2H), 3.43 (bs, 1H), 3.52 (s, 2H). MS (m/z): 231.4(M−1).

B. In Vitro Testing

The ability of negamycin or deoxynegamycin, or pharmaceutically acceptable salts, prodrugs or isomers thereof, to treat bacterial infection was measured by in vitro assay described below. The compounds were kept in DMSO at 10 mg/ml and diluted into the test medium for the assay.

The strains used for assaying negamycin include the following: Acinetobacter baumanii, Citrobacter freundii, Enterobacter aerogenes, Haemophilus influenzae, Moraxella catarrhalis, Staphylococcus aureus MRSA, Staphylococcus aureus GISA, Staphylococcus epidermis, Streptococcus pneumoniae PenR, Streptococcus pneumoniae PenS, Streptococcus pyogenes, Helicobacter pylori and E. faecium. The strains used for assaying deoxynegamycin include the following: Haemophilus influenzae, Staphylococcus aureus MRSA, Staphylococcus aureus GISA, Staphylococcus epidermis, Streptococcus pneumoniae PenR, Streptococcus pneumoniae PenS, Streptococcus pyogenes and E. facium.

(1) Liquid Medium, Nutrient Broth and Mueller-Hinton Broth with 50% Human Serum

### Preparation of Plates with Two-fold Dilutions of Test Compound

Compounds were suspended in a DMSO solution containing 10 mg/ml of compound. The appropriate amount of compound was added to a well of the first column of a 96-well microtiter plate containing 100 μl of Nutrient Broth (NB) or Mueller-Hinton Broth with 50% human serum (MHB+HS), for S. pneumoniae the NB medium was supplemented with 3 or 5% lysed horse blood. The rest of the wells in the row had 50 μl of the same medium in each well. After adding the compound to the first well and mixing thoroughly, 50 μl was taken out, and mixed to the second well of the row. This operation was repeated until the well of column 11 was reached and, after mixing, 50 μl were discarded. The last column was a control well that does not contain test compound. A concentration gradient of two-fold dilution was achieved by this method. A distinct compound was added to the first column well of the next row of the microtiter plate, and the procedure was repeated until all the rows are used. The plate was then ready for inoculation.

### Preparation of Inoculum

Bacterial strains were inoculated from a frozen stock on Mueller-Hinton Agar (Difco) or Blood Agar for S. pneumoniae and grown at 35° C. overnight. Cells were suspended in sterile saline to 0.5 McFarland standard. The inoculum was diluted approximately 1:1000 in the appropriate medium to reach an inoculum size of 105 cfu/ml (NB, NB supplemented with 3 or 5% lysed horse blood for S. pneumoniae, or MHB+HS). The diluted cell suspension was used to inoculate the microtiter plate containing two-fold dilution of the test compound. Each well of the microtiter plate received 50 μl of the cell suspension. The final volume was 100 μl (50 μl from the inoculum and 50 μl of the medium containing the compound).

### Incubation of the Plates

The plates were incubated at 35° C. for 16 to 20 hours, the minimal inhibitory concentration (MIC) was recorded and was defined as the lowest concentration of compound that there is no visible growth.

(2) Solid Medium, Mueller-Hinton Agar with 50% Human Serum

### Preparation of Petri Dishes for Assessing MIC in Solid Medium

Compounds were suspended in a DMSO solution containing 10 mg/ml of compound. The appropriate amount of compound was added to melted Mueller-Hinton Agar with 50% human serum (MHA+HS) at 50° C. The medium has been prepared by adding human serum to a melted Mueller-Hinton Agar (MHA) at two fold the concentration. MHA+HS has 50% human serum and normal strength of MHA. The appropriate concentration of compound was added to 4 ml of melted MHA+HS, and poured in a 6 cm diameter petri dish, and the agar was allowed to solidify at room temperature. A set of petri dishes, each dish containing half the concentration of compound of the previously poured dish, was made and used to assess the MIC in solid medium.

### Preparation of Inoculum

Bacterial strains were inoculated from a frozen stock on Mueller-Hinton Agar (Difco) or Blood Agar for S. pneumoniae and grown at 35° C. overnight. Cells are suspended in sterile saline to 0.5 McFarland standard. The inoculum was diluted approximately 1:10 in sterile saline water to obtain a bacterial suspension at 107 cfu/ml. This cell suspension was used to inoculate the petri dishes with varying concentration of test compound by applying 2 μl on the surface (approximately 104 cfu).

### Incubation of Plates

Petri dishes were incubated at 35° C. for 16 to 20 hours, the minimal inhibitory concentration (MIC) was recorded and was defined as the lowest concentration of compound that there is no visible growth.

### In Vitro Activity of Negamycin and Deoxynegamycin

Negamycin exhibited activity (i.e., MIC<128 μg/ml) against the following bacterial strains: Acinetobacter baumanii, Citrobacter freundii, Enterobacter aerogenes, Haemophilus influenzae, Moraxella catarrhalis, Staphylococcus aureus MRSA, Staphylococcus aureus GISA, Staphylococcus epidermis, Streptococcus pneumoniae PenR, Streptococcus pneumoniae PenS, Streptococcus pyogenes and Helicobacter pylori. Negamycin was inactive against E. faecium. Deoxynegamycin exhibited activity against the following bacterial strains: Haemophilus influenzae, Staphylococcus aureus MRSA, Staphylococcus aureus GISA, Staphylococcus epidermis, Streptococcus pneumoniae PenR, Streptococcus pneumoniae PenS and Streptococcus pyogenes. It was inactive against E. faecium.

B. In Vivo Testing

Escherichia coli ATCC 25922 was used to test the efficacy of negamycin (14) in a mouse septicemia model using six ICR male mice per group. Mice were inoculated intraperitoneally with 2×10⁵ cfu/mouse in 0.5 ml of Brain-Heart Infusion broth supplemented with 5% mucin. The compound was tested at 40, 20, 10, 5 and 2.5 mg/kg orally (PO) or at 20, 10, 5 and 2.5 mg/kg intravenously (IV) at 1 and 5 hours after bacterial inoculation. Controls included vehicle control (0.9% NaCl, 10 ml/kg) and an antibiotic control with ampicillin. Mortality was recorded for 7 days, and an ED₅₀ was determined by non-linear regression. Negamycin exhibited an ED₅₀ (mg/kg) of 4.1 (IV) and 15.1 (PO) in the model. Deoxynegamycin exhibited an ED₅₀ (mg/kg) of 4.8 (IV) in the model. Ampicillin, the control, exhibited an ED₅₀ of 6.8 (IV) and 27.6 (PO) under the same conditions.

The ED₅₀ (mg/kg) against S. Aureus was also determined to be 16.8 for negamycin.

From the foregoing description, various modifications and changes in the composition and method will occur to those skilled in the art. All such modifications coming within the scope of the appended claims are intended to be included therein.

What is claimed is:

1. A method of treating a bacterial infection, wherein the method comprises orally administering a pharmaceutical composition to an animal, and wherein the composition comprises a pharmaceutically acceptable excipient and an antibacterial effective amount of negamycin, or a pharmaceutically acceptable salt, prodrug or isomer thereof.

2. The method according to claim 1, wherein negamycin is orally administered to an animal.

3. The method according to claim 1, wherein the pharmaceutical composition further comprises at least one antibiotic in addition to negamycin.

4. The method according to claim 2, wherein from about 0.1 to about 100 mg/(kg animal)/day of negamycin is orally administered to an animal.

5. The method according to claim 3, wherein the added antibiotic is active against gram positive bacteria.

6. The method according to claim 4, wherein from about 0.1 to about 50 mg/(kg animal)/day of negamycin is orally administered to an animal.

7. The method according to claim 6, wherein from about 0.5 to about 20 mg/(kg animal)/day of negamycin is orally administered to an animal.

8. A method of treating a bacterial infection, wherein the method comprises intravenously administering a pharmaceutical composition to an animal, and wherein the composition comprises a pharmaceutically acceptable excipient and an antibacterial effective amount of deoxynegamycin, or a pharmaceutically acceptable salt, prodrug or isomer thereof.

9. The method according to claim 8, wherein deoxynegamycin is intravenously administered to an animal.

10. The method according to claim 8, wherein the pharmaceutical composition further comprises at least one antibiotic in addition to deoxynegamycin.

11. The method according to claim 9, wherein from about 0.1 to about 100 mg/(kg animal)/day of deoxynegamycin is orally administered to an animal.

12. The method according to claim 10, wherein the added antibiotic is active against gram positive bacteria.

13. The method according to claim 11, wherein from about 0.1 to about 50 mg/(kg animal)/day of deoxynegamycin is orally administered to an animal.

14. The method according to claim 13, wherein from about 0.5 to about 20 mg/(kg animal)/day of deoxynegamycin is orally administered to an animal.

15. A method of treating a bacterial infection, wherein the method comprises administering, to an animal, an antibacterial effective amount of negamycin or deoxynegamycin, or a pharmaceutically acceptable salt, prodrug or isomer thereof, and wherein the infecting bacteria are selected from a group of bacteria consisting of the following: Acinetobacter baumanii, Citrobacter freundii, Enterobacter aerogenes, Haemophilus influenzae, Moraxella catarrhalis, Staphylococcus aureus MRSA, Staphylococcus aureus GISA, Staphylococcus epidermis, Streptococcus pneumoniae PenR, Streptococcus pneumoniae PenS, Streptococcus pyogenes and Helicobacter pylori.

16. The method according to claim 15, wherein negamycin is administered to an animal.

17. The method according to claim 15, wherein deoxynegamycin is administered to an animal.

18. The method according to claim 15, wherein the infecting bacteria are selected from the group of bacteria consisting of the following: Acinetobacter baumanii, Citrobacter freundii and Enterobacter aerogenes.

19. The method according to claim 15, wherein the infecting bacteria are selected from the group of bacteria consisting of the following: Haemophilus influenzae and Moraxella catarrhalis.

20. The method according to claim 15, wherein the infecting bacteria are selected from the group of bacteria consisting of the following: Staphylococcus aureus MRSA, Staphylococcus aureus GISA and Staphylococcus epidermis.

21. The method according to claim 15, wherein the infecting bacteria are selected from the group of bacteria consisting of the following: Streptococcus pneumoniae PenR, Streptococcus pneumoniae PenS and Streptococcus pyogenes.

22. The method according to claim 16, wherein from about 0.1 to about 50 mg/(kg animal)/day of negamycin is administered to an animal.

23. The method according to claim 17, wherein from about 0.1 to about 50 mg/(kg animal)/day of deoxynegamycin is administered to an animal.

24. The method according to claim 22, wherein from about 0.5 to about 20 mg/(kg animal)/day of negamycin is administered to an animal.

25. The method according to claim 23, wherein from about 0.5 to about 20 mg/(kg animal)/day of deoxynegamycin is administered to an animal.
